(19) United States
(12) Patent Application Publication
(10) Pub. No.: US 2017/0188941 A1
(43) Pub. Date: Jul. 6, 2017

(54) DEVICE AND METHOD FOR NON-INVASIVEL Y MONITORING A SEDATED OR ANESTHETIZED PERSON

(71) Applicant: Bernhard BRINKHAUS, Oetwil an der Limmat (CH)

(72) Inventor: Bernhard BRINKHAUS, Oetwil an der Limmat (CH)

(21) Appl. No.: 15/125,062

(22) PCT Filed: Mar. 16, 2015
(86) PCT No.: PCT/EP15/55439

§ 371 Date: Feb. 6, 2017

(30) Foreign Application Priority Data
Mar. 14, 2014 (EP) 14160095.7

(51) Int. Cl. A61B 5/00

(52) U.S. Cl. CPC A61B 5/4824; A61B 5/4277; A61F 2007/0295

(57) ABSTRACT

The invention relates to a device (1) for non-invasively monitoring a sedated or anesthetized person (10) comprising a pain-stimulus-generating device (7), a measuring device (2) for capturing a saliva amylase value (S(t)) of the person (10), a control device (4), and an output device (5), wherein the control device (4) is designed in such a way that the control device controls the pain-stimulus-generating device (7) in order to produce a thermal pain stimulus (R), and that, alter the pain stimulus (R) has been produced, the control device (4) captures the saliva amylase value (S(t)) and outputs an output value (A) by means of the output device (5), wherein the output value (A) is the saliva amylase value (S(t)) or a value dependent on the saliva amylase value (S(t)).

The invention relates to a device and to a method for noninvasively monitoring a sedated or anesthetized person.

### PRIOR ART

For a multiplicity of surgical procedures on the human body, it is necessary to ensure that the sensation of pain is reduced during the surgical procedure. Examples of known ways to reduce the sensation of pain are:
local anesthesia;
minimal sedation, also referred to as twilight sleep;
deep sedation, also referred to as analgosedation or deep sleep;
general anesthesia.

Local anesthesia is one form of anesthesia. It is defined as local suppression of pain in the region of nerve endings or of pathways, without interfering with consciousness. Owing to targeted administration of anesthetics, usually local anesthetics, it brings about the temporary, reversible inhibition of function of selected nerves and in doing so leads to insensitiveness and absence of pain.

The term sedation refers to the attenuating of functions of the central nervous system by means of a tranquilizer, also referred to as a sedative if a painkiller, also referred to as an analgesic, is administered at the same time, this is called analgosedation. The transition from sedation to general anesthesia is fluid. In the case of general anesthesia, the patient is no longer arousable.

An overview of the sedation-general anesthesia continuum is shown below (American Society or Anesthesiologists).

| | Minimal | Deep | General |
|---|---|---|---|
| | sedation | sedation | anesthesia |
| Responsiveness | Arousable | Arousable | Unarousable |
| | | with | |
| | | stimulation | |
| | | or pain | |
| | | stimulus | |
| Airway | Not | May be | Generally |
| intervention | necessary | necessary | necessary |
| Spontaneous | Adequate | May be | Generally no |
| ventilation | | reduced | longer |
| | | | present |
| Cardiovascular | Generally | Generally | May be |
| system | unimpaired | unimpaired | impaired |

[From: Continuum of depth of sedation, definition of general anesthesia, and levels of sedation/analgesia. American. Society of Anesthesiologists Standards, Guidelines, and Statements, Oct. 27, 2004]

General anesthesia is one form of anesthesia, the goal of which is to suppress the patient's consciousness and pain sensation in order to be able to carry out diagnostic or therapeutic surgical procedures, especially operations, and to provide optimal conditions therefor both for the patient and for the physician. To this end, one or more general anesthetics, which have an effect in the central nervous system, are administered. When this is done, the patient is not arousable, in contrast to local anesthesia in which the suppression of pain via the blockade of nerve fibers covers only individual regions of the body.

General anesthesia is usually carried out under planned circumstances as part of an operation. In the case of endotracheal intubation for airway management in emergency care and intensive care, general anesthesia can likewise be used; to continue the ventilation therapy, sedation is subsequently sufficient.

General anesthesia has certain advantages. The patient is looked after intensively by an anesthetist and permanently monitored in order “to stay alive”. Therefore, there is only a very small risk that the patient is awake during the operation or feels pain. Moreover, the duration of the general anesthesia can be controlled, meaning that as much time as necessary is available to the surgeon for the surgical procedure.

However, general anesthesia also has disadvantages. These include the need for an empty stomach before the surgical procedure, which can weaken the patient and which, moreover, can have a long-lasting adverse effect on pain behavior after the operation. Moreover, considerable effort in terms of medicaments and apparatuses is required, in addition to the requirement that a second physician monitors the general anesthesia as an anesthetist. General anesthesia is therefore relatively cost-intensive.

Analgosedation likewise has certain advantages. A patient does not need to be on an empty stomach, does not need to undergo a preliminary examination and can therefore be treated straightaway as an outpatient. The effort for analgosedation in terms of apparatuses and. medicaments is substantially lower and can also be carried out by a surgeon trained in anesthesiology. The substantially lower effort also means distinctly lower stress for the patient, and so unstressable patients with the subjective effect of general anesthesia can be treated surgically too.

One problem which may occasionally occur in local anesthesia and in general anesthesia, but which predominantly occurs in analgosedation, is the fact that it is not ensured that pain sensation is suppressed during the surgical procedure, and so it is still possible for pain sensation to occur even during analgosedation. Therefore, a substantial disadvantage of analgosedation is a possible restlessness which, triggered by pain stimuli of the surgical procedure, forces the termination of the operation in extreme cases, since the subconscious patient still perceives the pain stimuli of the operation despite deep sleep.

However, the problem of a reliable suppression of pain has also not yet been solved in the field of general anesthesia. Since the beginning of general anesthesia procedures, one aim has been to monitor the depth of anesthesia. In this connection, an excessively “superficial” anesthesia with insufficient suppression. of pain is just as undesired as an excessively “deep” general anesthesia with corresponding hemodynamic impairment, delayed postoperative arousal, prolonged monitoring times and unnecessarily high anesthetic consumption. Therefore, a personalized anesthesia management would be especially advantageous. This is also meaningful from economic points of view for reducing costs, but in particular for reducing personnel engagement and surgical theater occupancy as a result of unnecessarily long finishing times. Nowadays, depth of anesthesia is individually controlled especially by monitoring hemodynamics and. vegetative changes, the most important reference points being blood pressure and heart rate behavior, and also spontaneous movements of the patient, lacrimation, perspiration or pupillary light reflex. If opioids are used, as is customary in modern anesthetic techniques, clinical assessment of the depth of anesthesia is additionally hampered because hemodynamic lapses and vegetative signs may be completely missing, and nevertheless the reporting of anesthesia awareness by the patient cannot be ruled out.

Document DE10 2009 053 256 A1 discloses a method for determining the analgesia level of a sedated or general-anesthetized individual by using an evoked pain-specific reflex response. Said method uses a stimulation signal in order to bring about the triggering of a pain-specific reflex in an individual. The pain-specific reflex used is either the defensive and flight reflex, such as the eye blink reflex, or the flexor reflex, especially of the lower extremities. The RIII reflex used in this connection is, as component of the nociceptive flexor reflex (NFR), a polysynaptic spinal retraction reflex which is triggered by stimulation of nociceptive afferent nerves. The reflex response generated by the stimulation signal appears within a period from 1 ms to 1 s after the stimulation. What is disadvantageous about this method is the fact that the individual probability of predicting a response or the nonappearance of a response to pain stimuli is not satisfactory. The method is therefore relatively unreliable.

Document JP 2010 081950A discloses a method for detecting pain of a sedated patient during an endoscopic submucosal dissection (ESD), involving collecting a saliva sample from the patient and measuring the amylase in the saliva sample. An increase in the amylase value is used as an indication of an increase in pain. A disadvantage of said method is the fact that only an indication of an increase in pain can be established, but without obtaining a specific indication as to why the pain has increased. Thus, for example, it remains open as to whether the inflicted pain per se became greater, or whether the effect of a tranquilizer is subsiding. Therefore, a physician, proceeding from an increase in the amylase value, cannot deduce clear action steps.

### DESCRIPTION OF THE INVENTION

It is an object of the invention a device and a method for the improved, noninvasive monitoring of a sedated or anesthetized person.

In particular, it is a further object of the present invention a device and a method for noninvasively determining the depth of sedation or the depth of general anesthesia of a sedated or anesthetized person.

This object is achieved by a device having the features of claim 1. Dependent claims 2 to 10 concern further, advantageous embodiments. The object is further achieved by a method having the features of claim 11. Dependent claims 12 to 20 concern further, advantageous method steps.

The object is achieved in particular by a device for noninvasively monitoring a sedated or anesthetized person, comprising a pain-stimulus generation device, comprising a measurement device for capturing a salivary amylase value of the person, comprising a control device and also comprising an output device, wherein the control device is designed such that it controls the pain-stimulus generation device in order to generate a pain stimulus and that, after effected. generation of the pain stimulus, the control device captures the salivary amylase value and outputs an output value via the output device, wherein the output value is the salivary amylase value or value dependent on the salivary amylase value.

The object is achieved in particular by a method for noninvasively monitoring a sedated or anesthetized person, by delivering a pain stimulus to the person, by measuring a salivary amylase value of the person after effected delivery of the pain stimulus, and by outputting the measured salivary amylase value or a value dependent on the salivary amylase value.

The device according to the invention or the method. according to the invention measures the salivary amylase activity or the salivary amylase concentration, also referred to hereinafter as salivary amylase value, especially during the time period over which the person is sedated or anesthetized.

It is known from the literature that there is a rise in α-amylase in saliva in humans following mental stress. However, it has now also been found that the salivary amylase value is moreover a bioindicator of pain in a person. In particular, it has been found that the salivary amylase value can also rise in sedated or anesthetized persons when they are exposed to severe pain, or when the sedation or the anesthesia is not sufficiently deep. In such a case, the pain activates the autonomic nervous system.

The device according to the invention and the method according to the invention therefore have the advantage that the salivary amylase value can be measured noninvasively in a sedated or anesthetized person, the measured salivary amylase value or the output value generated on the basis of the salivary amylase value being functionally linked to the pain which has occurred or to a depth of general anesthesia or sedation. With the aid of said output value and on the basis of his professional expertise, a physician can therefore dictate necessary actions in order, for example, to reduce the pain, to take a break, to administer painkillers, or to deepen the general anesthesia.

Salivary amylase, also referred to as α-amylase 1 or ptyalin, is understood to mean three enzyme isoforms produced in saliva by humans. The enzyme is that enzyme in all living creatures which is able to cleave storage carbohydrates such as starch and glycogen into its constituents via the splitting of 1,4-α-D-glycosidic bonds. For many vertebrates, including in humans, carbohydrate digestion starts with the production of the enzyme in saliva α-Amylase (EC 3.2.1.1) cleaves the α(1-4) glycosidic bond in amylose. This yields dextrins and, therefrom, maltose, glucose and branched oligosaccharides. In humans, there are five isoforms of α-amylase, the genes of which are named AMY1A, AMY1B, AMY1C (all three called salivary amylase) and AMY2A and AMY2B (both pancreatic amylase).

The device according to the invention comprises a pain-stimulus generation device which is applied to the patient in order to generate a pain stimulus by means of a temperature stimulus or to bring about a pain-stimulus stimulation. The course of the salivary amylase value is preferably monitored within a predetermined time frame immediately after the generation of the pain stimulus, for example within a time frame from 10 seconds to 5 minutes after the pain-stimulus stimulation, in order to establish whether there is an increase in the salivary amylase value owing to the pain stimulus. If the increase in the salivary amylase value meets certain criteria and, in particular, takes place within the stated time frame, it can be inferred therefrom that a pain stimulus, caused by the pain-stimulus generation device, has occurred at the patient, which pain stimulus led to an activation of the autonomic nervous system. If there is no increase in the salivary amylase value following the pain stimulus inflicted on the patient, it can be inferred therefrom that, the patient is under deep sedation or deep general anesthesia such that the inflicted pain does not result in an increased activity of the autonomic nervous system. The device according to the invention or the method according to the invention therefore makes it possible to monitor the sedation or the general anesthesia, or the depth of sedation or general anesthesia, of a person and, in particular, to monitor it on an individual basis. In an especially advantageous embodiment, the device according to the invention or the method according to the invention allows, as a result of the monitoring of the pain which has occurred or the monitoring of the depth of sedation or general anesthesia, a personalized anesthesia management, or a personalized course of the analgosedation of a patient. Therefore, a physician can administer personalized doses of sedatives, painkillers or general anesthetics to a patient during an operation in order to keep the patient in an advantageous depth of sedation or general anesthesia. In an advantageous embodiment, the device according to the invention additionally comprises a dispensing device which proposes to a physician at least the quantity and/or the nature of the medicament to be administered and preferably also the time of the administration on the basis of the measured salivary amylase values. In a further advantageous embodiment, the dispensing device is designed such that it administers the medicaments automatically, preferably after the physician has confirmed the proposed medicaments, the dosage and the time.

In a further advantageous method step, an individual sensitivity to pain of a patient is captured prior to an operation on the patient, by exposing the patient to a single pain stimulus and preferably, in succession, multiple pain stimuli of differing strength and by measuring the salivary amylase value occurring in the course of this, and so, before the operation, there is for each patient an individual relationship between pain stimulus and the corresponding salivary amylase values or a relationship between the temperature of the pain stimuli and the salivary amylase values brought about thereby. This is especially advantageous because people react differently to pain stimuli such as temperature stimuli from individual to individual. People have sensitivity profiles which differ from individual to individual. In a further advantageous method step, not only the temperature stimulus level and the salivary amylase value but also additionally a subjective pain intensity are measured or queried and saved prior to an operation during the capture of the sensitivity profile. A stored individual sensitivity profile of this kind comprising measurement values of the salivary amylase value and of the subjective pain intensity as a function of the level or temperature of the temperature stimulus makes it possible to infer the subjective pain intensity during an operation on the basis of the salivary amylase value measured during the operation and to therefore display the subjective pain intensity during the operation as an output value or to propose further actions on the basis of the output value.

The sympathetic nervous system is part of the autonomic nervous system. The activation of the sympathetic nervous system due to the temperature stimulus of the pain-stimulus generation device results in an increase in the concentration of the enzyme alpha-amylase in saliva. An increase in alpha-amylase in saliva is detectable in particular from 10 seconds to 1 minute after excitation of the sympathetic nervous system or after the stimulation with the temperature stimulus. Therefore, the time frame for monitoring the alpha-amylase increase caused by the stimulation preferably starts at 10 seconds to 1 minute after stimulation has been effected. It is also evident therefrom that the reflex signal which is measured in the earlier-cited document DE 102009053256A1 and which is registered within a time frame from 1 millisecond to 1 second after the stimulation monitors a completely different response of the human body, namely the RIII reflex, a polysynaptic spinal retraction reflex.

The device according to the invention and the method according to the invention are suitable for noninvasively monitoring patients under local anesthesia, minimal sedation, deep sedation or under general anesthesia.

### BRIEF DESCRIPTION OF THE DRAWINGS

The invention will be elucidated in detail below by means of a multiplicity of exemplary embodiments. The figures depicted show:

FIG. 1 first exemplary embodiment of the monitoring device;

FIG. 2 an example of the course of the salivary amylase value as a function of time during an operation;

FIG. 3 an individual course of the salivary amylase value as a function of the temperature of a temperature stimulus;

FIG. 2 an individual course of the salivary amylase value as a function of time and of temperature;

FIG. 5 a temporal sequence before, during and after an operation;

FIG. 6 a pain stimulus as a function of time;

FIG. 7 a further example of the course of the salivary amylase value as a function of time during an operation;

FIG. 8 a further example of the course of the pain stimulus and of the salivary amylase value as a function of time during an operation;

FIG. 9 an extract from a course of the pain stimulus and of the salivary amylase value as a function of time during an operation;

FIG. 10 an extract from a course of the pain stimulus and of the course of two salivary amylase values as a function of time during an operation;

FIG. 11 a further extract from a course of the pain stimulus and of the salivary amylase value as a function of time during an operation;

FIGS. 12 a further extract from a course of the pain stimulus and of the salivary amylase value as a function of time during an operation;

FIG. 13 a further example of a course of the salivary amylase value as a function of time during an operation;

FIG. 14 second exemplary embodiment of the monitoring device;

FIG. 15 an individual course of the sensitivity to pain, or an individual course of the salivary amylase value and of the subjective pain intensity as a function of temperature and of time;

FIG. 16 the individual course of the sensitivity to pain or an individual course of the salivary amylase value and of the subjective pain intensity as a function of temperature and of time from two different persons.

In the drawings, the same parts are always provided with the same reference signs.

### WAYS OF CARRYING OUT THE INVENTION

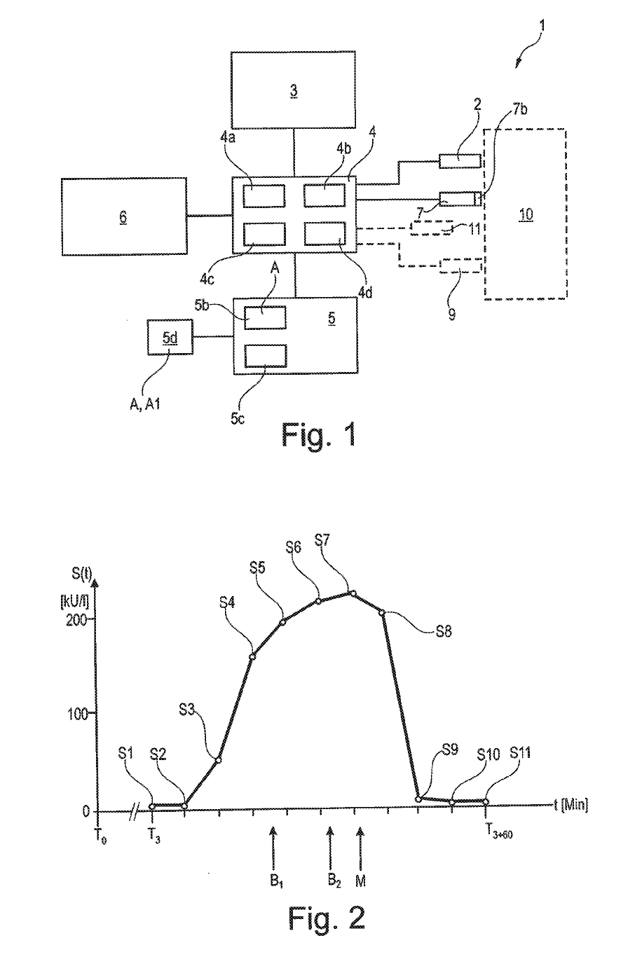

FIG. 1 shows a first exemplary embodiment of a device 1 for monitoring a sedated or anesthetized person 10 or a patient 10. The monitoring device 1 comprises a pain-stimulus generation device 7 and a measurement device 2 for capturing a salivary amylase value S of the person 10. The monitoring device 1 additionally comprises a control device 4 which is connected to the pain-stimulus generation device 7 and the measurement device in a signal-transmitting manner, for example via electrical lines. The monitoring device 1 additionally comprises a storage device 3 for individual personal data, a storage device 6 for further data arising during the monitoring, and an output device 5, which are connected to the control device 4 in a signal-transmitting manner. The control device 4 comprises, inter alia, a signal-processing unit 4a, a stimulus control unit 4b, a decision unit 4c and a calculator 4d. The output device 5 preferably comprises a screen 5b for outputting an output value A, an input device 5c, for example a keyboard, and preferably additionally a display device 5d for outputting an alarm value A1. The pain-stimulus generation device 7 comprises a heatable, preferably electrically heatable, device 7b, especially an electrical resistor, it being possible to apply the device or a heated part of the device to the skin of the patient 10. The pain-stimulus generation device 7 must be arranged such that it can exert a thermal stimulus on the skin of the patient 10, the pain-stimulus generation device 7 advantageously coining directly into contact with the skin and lying flat against said skin. However, it has also been found to be advantageous, for example for reasons of hygiene, to provide an intermediate layer between the pain-stimulus generation device 7 and the skin of the patient 10, for example a thin piece of plastic.

In an advantageous embodiment, it is additionally possible to connect further input or output means to the control device 4, for example an input device 11 or a motion sensor 9.

The pain-stimulus generation device 7 serves to inflict on the person 10 a thermal stimulus which is defined and reproducible and which can also be sensed as a pain stimulus from a certain intensity. The present invention uses thermally acting means for pain-stimulus generation. Thermal pain-stimulus generation has been found to be particularly advantageous because the pain stimulus is easily reproducible. A thermal stimulation probe, as disclosed in WO13168168A1, is, for example, suitable for thermal pain-stimulus generation. Such a pain-stimulus generation device is small and compact, and allows a controlled, reproducible thermal stimulation, it being possible to administer the pain stimulus in a multiplicity of possible courses as a function of time. In an advantageous embodiment, the pain-stimulus generation device comprises, as disclosed in document WO13168168A1, in addition a Peltier element for cooling purposes. In an advantageous embodiment, the pain-stimulus generation device comprises an electrically heatable resistor and preferably additionally a thermoelectric cooler designed as a Peltier element. Such a pain-stimulus generation device 7 allows a precisely controlled, reproducible thermal stimulation as a function of time, having a defined temperature course as a function of time and having a temperature rise within the range from 0.5° C. to 2° C. per second, and, if a Peltier element is provided, also having a defined temperature drop within the range from 0.5° C. to 2° C per second. Preferably, use is made of a temperature rise of about 1° C. per second and, if provided, also a temperature drop of about 1° C. per second. The pain-stimulus generation device 7 or the device 7b advantageously comprises a temperature sensor so that the temperature released by the pain-stimulus generation device 7 is captured, and thus the heating and, if necessary also the cooling, is effected such that the temperature released by the pain-stimulus generation device 7 corresponds to a nominal value or nominal value course predetermined by the control device 4. The pain-stimulus generation device is advantageously directly applied to the skin of a person, preferably always at the same spot, for example at a spot on the upper or lower arm, or on the leg.

The salivary amylase value is determinable in a noninvasive manner via a measurement device 2, for example using a measurement device having the name “Salivary amylase monitor®” from NIPRO Inc., Osaka, Japan.

Depending on the design and the operating mode of the measurement device 2, said device measures the salivary amylase value S only once in each case, or measures the salivary amylase value S(t) at discrete points in time, for example every 6 minutes, or, for example, even continuously or at short intervals, for example every 10 to 30 seconds.

FIG. 2 shows, along the time axis t, a temporal extract from a course of an operation, the depicted extract depicting the course from time T3 up to time T3 60 minutes. The patient is in an analgosedation state and the depth of sedation is monitored along the time axis t by means of classic subjective criteria and physiological parameters, such as, for example, facial expression, movement, heart rate, blood pressure, respiratory rate, lacrimation and/or sweating. FIG. 2 depicts, along the time axis t, the monitoring of movement B. During the course of the operation, a movement of the patient is established at the time described by B₁. Subsequently, a further, more violent movement of the patient is established at the time described by B₂. The monitoring physician infers from the movement which occurred that the patient is sensing pain, and that the sedation needs to be deepened, and so the physician administers an analgesic, preferably a highly effective painkiller, at time M. After this administration, no more body movements are established. until the end of the depicted observation period, i.e., T3+60 minutes.

FIG. 2 additionally shows, in the vertical direction, the salivary amylase value S(t) measured during the operation as a function of time, or the salivary amylase activity in kU/l. The monitoring device 1 depicted in FIG. 1 can be used to measure the salivary amylase value S(t), although the pain-stimulus generation device 7 was not used in the course depicted in FIG. 2 and therefore did not come into use. Therefore, only the salivary amylase value S(t) was measured as a function of time. The salivary amylase values S1, S2, . . . S11 were measured every 6 minutes in the depicted example. From the course of the salivary amylase values S1 . . . S11 as a function of time, it is evident that the patient already exhibits a rise in value at measurement value S3, it being possible to infer therefrom that said patient sensed a pain stimulus at measurement value S3, and the pain stimulus continuously rises up to measurement value S7, resulting in the movements at time B1 and. B2. After the administration of the analgesic N, it is evident from the measurement values S9, S10 and S11 that the levels of pain were strongly reduced. Owing to the measurement of the salivary amylase value during the operation, it was identified that the salivary amylase value is suitable as an indicator for the pain stimuli or levels of pain that occurred during the operation.

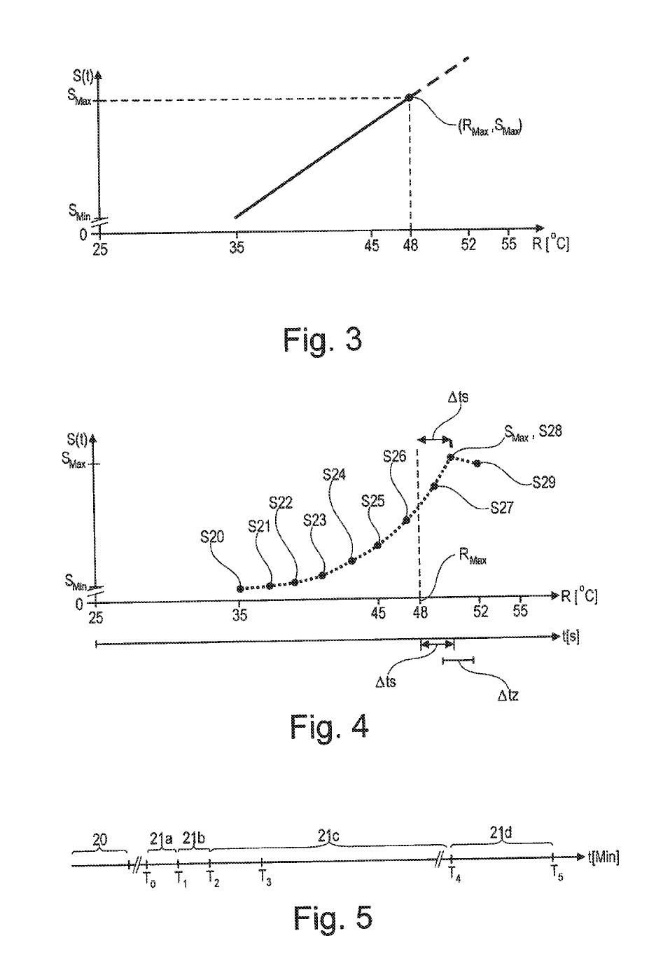

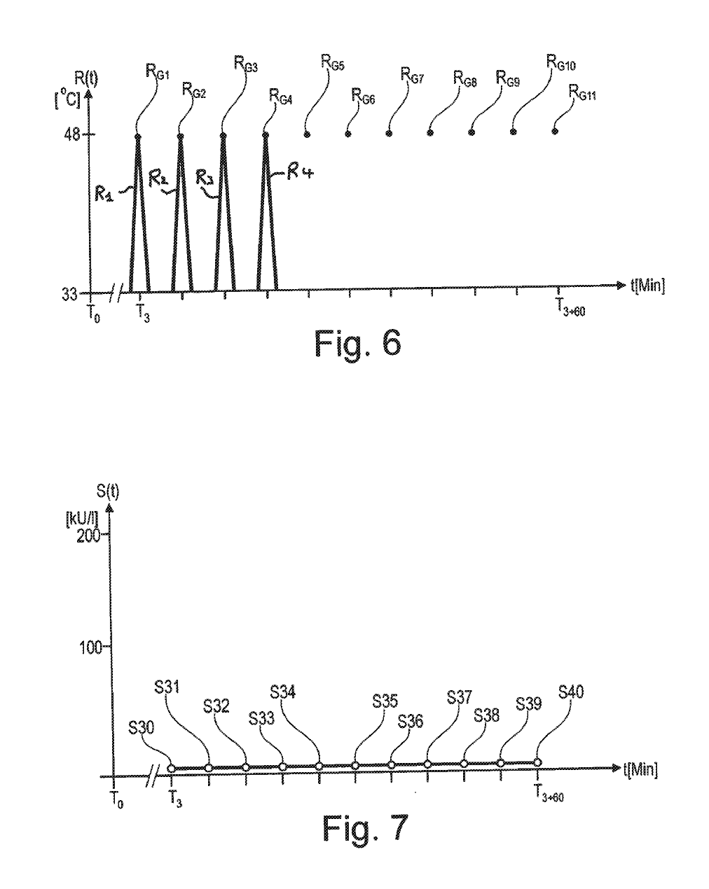

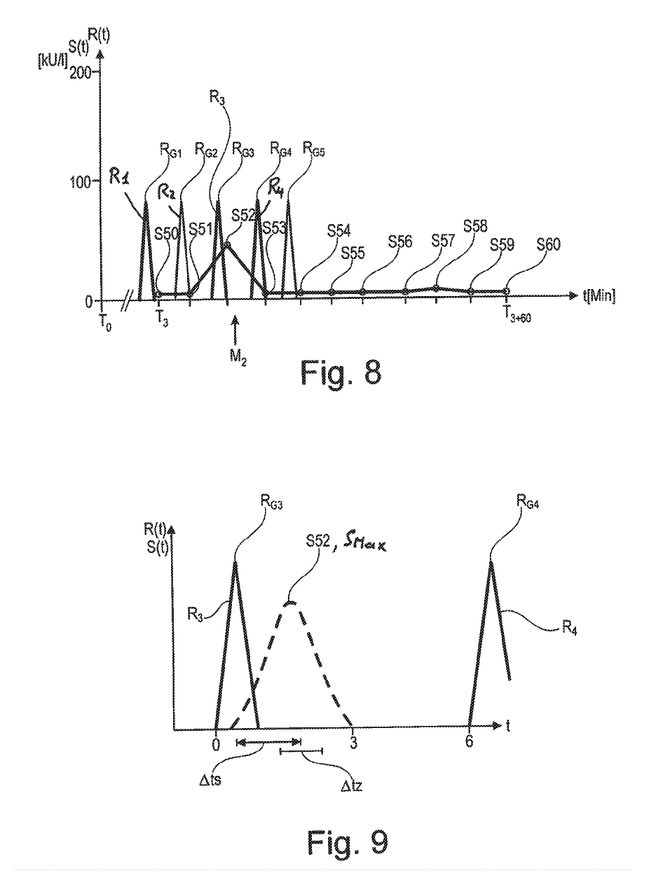

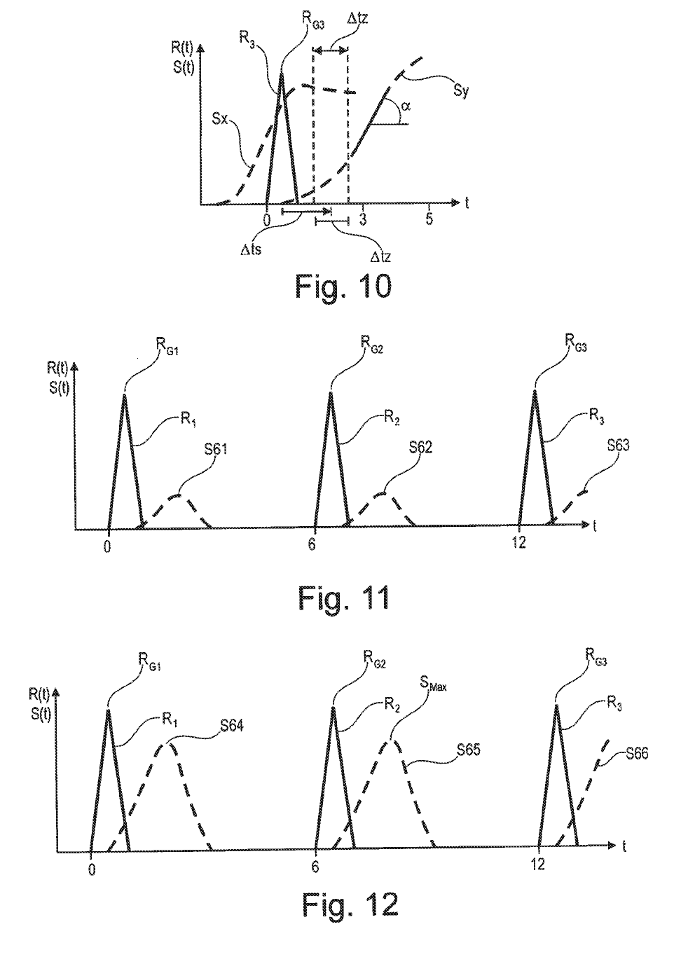

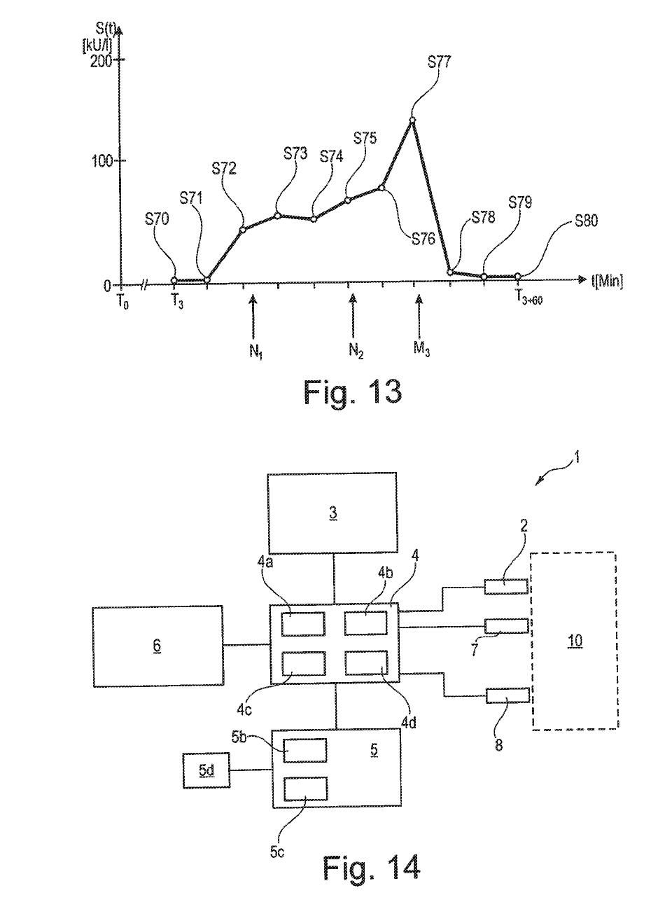

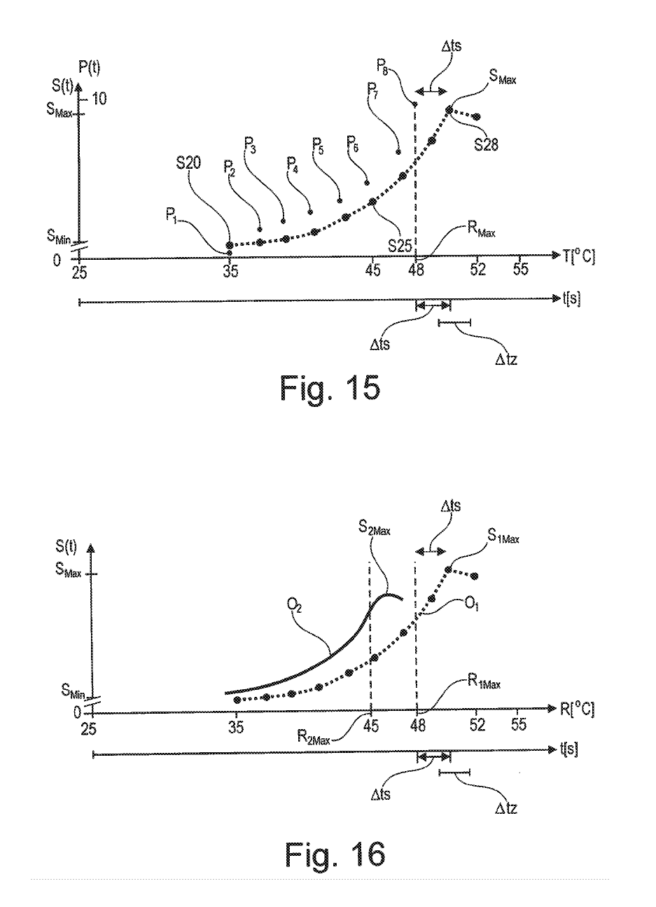

FIG. 3 shows an individual course of the salivary amylase value S as a function of the temperature T of a temperature stimulus, the temperature stimulus generating a pain stimulus R at relatively high temperatures. The temperature stimulus was generated using a thermal stimulation probe which rested on the skin at the inner surface of the lower arm. The relationship between the temperature T of the pain stimulus R and the resulting salivary amylase value S (T) is depicted in idealized form as a straight line in FIG. 3. It was established that there is a functional relationship between the temperature T and the salivary amylase value S, and that the salivary amylase value S also rises with increasing temperature T. In order to safely rule out damage to the skin, the temperature T is increased up to a maximum of 52° C. Above this value, there is the risk that the skin could be damaged owing to the high temperature.

FIG. 4 shows, in one example, the capture of an individual sensitivity to pain of a patient. To this end, a thermal stimulator is fastened to the lower arm of a patient, for example in such a way that the stimulation surface of the thermal stimulator rests on the inner surface of the lower arm. The thermal stimulator can be applied anywhere on the patient, preferably on the lower arm, on the foot or on a finger. Using the monitoring device 1 depicted in FIG. 1, the temperature of the thermal stimulator 7 is increased, and in addition the salivary amylase value is measured using the measurement device 2. In the case of the measurement of sensitivity to pain that is depicted in FIG. 4, the thermal stimulator 7 starts with a temperature of 25° C., with the temperature being increased at a rate of increase of, for example, 1° C. per 2 seconds. Owing to this relationship between temperature and time, FIG. 4 depicts both the temperature and the time. The temperature T rises increasingly in FIG. 4, with the thermal stimulus being increasingly painful from a temperature of about 40° C., the patient aborting the measurement by pressing the switch 11, at 48° C in the exemplary embodiment depicted, because the patient is now feeling uncomfortably severe pain. The temperature at abortion of the measurement is referred to as R_{Max}. The salivary amylase value rises in a delayed manner in relation to the temperature, the highest amylase value S_{Max} being measured with a time delay of Δts after the abortion of the measurement. The time delay Δts is, for example, 2 minutes, it being possible for the highest amylase value S_{Max} to occur within a time frame Δtz of, for example, ±2 minutes after the time delay Δts. Particularly advantageously, the time frame is selected such that the time frame opens at an opening time tₘᵢₙ, the opening time tₘᵢₙ being within a range from 10 seconds to 60 seconds after the occurrence of the value Rₘₐₓ or the stimulus threshold R_{g}. The time frame is advantageously closed 4 to 5 minutes after the occurrence of the value R_{Max} or of the stimulus threshold R_{g}. The salivary amylase value is preferably measured within this time frame in order to ensure that the measured salivary amylase value represents a response to the thermal stimulus. The value Sₘₐₓ and in particular the value R_{Max} are person-specific, individual values which are stored in the storage device 3, and express the sensitivity to pain of a patient. Pain-sensitive patients already sense a high level of pain at lower temperatures, whereas patients who are less pain-sensitive only sense pain at higher temperatures.

The value S_{Max} can be generated using different temperature stimuli proceeding as a function of time. In a further exemplary, possible method, the patient indicates, by pressing the switch 11, that the temperature stimulus is generating very mild levels of pain, whereupon the temperature is increased again, for example by 2° C., generating a severe pain stimulus. Thereafter, the temperature is preferably actively reduced, for example using a Peltier element. The temperature is never increased above 52° C in order to rule out damage to skin. The maximum amylase value Sₘₐₓ is subsequently measured after a time delay of Δts, for example after 2 to 3 minutes. The value S_{Max} and the value R_{Max} are subsequently stored as person-specific, individual values, with R_{Max} corresponding to the patient-triggered temperature value plus 2° C.

As depicted in FIG. 4, it is also possible to measure the entire curve profile of the salivary amylase value S(t) as a function of time t, i.e., all measurement points S20, S21 . . . S28, S29 are captured. Capturing the entire curve profile has the advantage that the measurement, especially the measurement of the maximum amylase value S_{Max}, is more accurate and is more reproducible.

FIG. 5 shows the sequence, before, during and after an operation, as a function of time. Several days before the operation, an operation preparation 20 usually already takes place, during which the measurement depicted in FIG. 4 is preferably carried out. In FIG. 5, the entire surgical phase starts at time T₀ and ends at time T₅, with a presurgical phase 21a taking place up to time T₁, during which the patient is prepared for the operation. This is followed by a sedation or general anesthetization phase up to time T₂. The operation or the surgical procedure 21c takes place between times T₂ and T₄. The wake-up phase 21d takes place between times T₄ and T₅.

The measurement of sensitivity to pain that is depicted in FIG. 4 can also take place during the presurgical phase 21a, especially in the case of an operation arranged at short notice, before which the patient is conscious and is highly responsive. Moreover, there is also the possibility of carrying out a measurement of sensitivity to pain once again in the presurgical phase 21a in order to compare the values with a measurement of sensitivity to pain that was carried out in advance, for example carried out during the operation preparation 20. This makes it possible to check whether the patient has the same values, or whether the values have substantially changed, for example owing to the mental stress associated with the operation preparation. In such a case, the values determined during the presurgical phase 21a would be preferentially used during the operation however, there is also the possibility of completely dispensing with the measurement of sensitivity to pain, especially in the case of an emergency operation, especially when the patient is no longer responsive before the operation or in the presurgical phase 21a. In such a situation, the maximum stimulus value is not individually determined; instead, a stimulus value R_{Max} is defined, for example a value of 48° C., it being necessary for the value always to be below 52° C.

The noninvasive monitoring of the sedated or anesthetized patient can take place in different ways during the operation. Some exemplary embodiments of noninvasive monitoring will be described below. No later than the start of the surgical procedure 21c, though preferably as early as during the presurgical phase 21a or the sedation or general anesthetization phase 21b, the pain-stimulus generation device 7, a thermal stimulator, is applied to the patient so that the part generating the thermal stimulus rests on the skin. An aspiration part for aspirating saliva, which part forms part of the measurement device 2, is placed into the mouth of the patient so that saliva can be aspirated from the mouth via a tube and fed to a consecutively arranged sensor of the measurement device 2, the sensor of the measurement device 2 measuring the salivary amylase value or the salivary amylase concentration. During the operation, the patient is naturally also treated with the other instruments, probes, etc that are required for an operation. However, for the operation of the in device 1, what is required is only some saliva from the patient and also the thermal stimulator 7 in contact with the patient's skin, and so the inventive device 1 can monitor the patient noninvasively, i.e., without any surgical interventions into the human body.

FIG. 6 shows the pain stimulus or thermal stimulus R(t), brought about by the thermal stimulator 7 on the patient, as a function of time. The time axis shows a time course during the operation, namely from time T3 up to time T3+60 minutes. The thermal stimulator 7 generates a temperature stimulus proceeding in a ramped. manner, which stimulus starts at 33° C. and rises at a rate of 1° C. per two seconds up to a value of 48° C., the stimulus threshold R_{G}, and then drops down to the value of 33° C. at a rate of again 1° C. per two seconds. A single thermal stimulus cycle of this kind, including rise and drop, i.e., starting from the temperature value of 33° C. to the stimulus threshold of 48° C. and back to the temperature value of 33° C., therefore lasts 1 minute in total, and therefore has a cycle duration of 1 minute. In a preferred embodiment, the cycle duration is constant throughout the monitoring. An advantageous cycle duration is within the range from 1 to 15 minutes. In the depicted exemplary embodiment, the thermal stimulus cycles R_{G1} . . . R_{G11} also comprising the stimulus thresholds R_{G1}, R_{G2}, R_{G3}, R_{G4} are effected at regular intervals, or the thermal stimulus cycles are repeated at regular intervals, every 6 minutes in the depicted example, and so the repetition rate is 6 minutes, the entire temperature course including temperature rise and temperature drop only being depicted in detail in the first four thermal stimulus cycles R₁, R₂, R₃, R₄, including the stimulus thresholds R_{G1}, R_{G2}, R_{G3}, R_{G4}, the entire course not being depicted anymore with respect to the remaining thermal stimulus cycles but only the stimulus thresholds R_{G5}, R_{G6}, R_{G7}, R_{G8} . . . being depicted, said thermal stimulus cycles however having the same temperature course as the first four thermal stimulus cycles R₁, R₂, R₃, R₄. Preferably, the thermal stimulus depicted in FIG. 6 is exerted on the patient over the entire duration of the operation 21c, i.e., from time T₂ up to time T₄. The repetition rate is advantageously within the range between 5 minutes and 60 minutes.

FIG. 7 shows the salivary amylase value S(t) as a function of time over the operation, namely from time T₃ to time T₃+60 minutes. The patient is under deep general anesthesia. The salivary amylase value S(t) is measured every 6 minutes, giving rise to the measurement points S30 . . . S40. The measured salivary amylase values are within the range of 0, indicating that the patient is not responding at all to the thermal stimulus cycles depicted in FIG. 6 or to the stimulus thresholds R_{G1} . . . R_{G11}, it being possible to infer therefrom that the patient continues to be under deep general anesthesia.

In contrast, FIG. 8 shows a salivary amylase value S(t) as a function of time, in which the patient feels a sensation of pain. FIG. 8 shows the measured salivary amylase values S₅₀ . . . S₆₀ over the operation, namely from time T₃ up to time T₃+60 minutes. The patient is analgosedated. The stimulus thresholds R_{G1} . . . R_{G11} or the thermal stimulus cycles R₁, R₂, R₃, R₄ . . . thereof are exerted on the patient in a regular sequence, with FIG. 8 depicting only the first five thermal stimulus cycles R₁, R₂, R₃, R₄ with stimulus thresholds R_{G1} . . . R_{G5}. After each thermal stimulus cycle, the corresponding salivary amylase values S₅₀ . . . S₆₀ that occur with a time delay with respect to the thermal stimulus or the stimulus threshold are subsequently measured. The thermal stimulus cycles are repeated every 6 minutes, and the salivary amylase values are measured about 1 to 3 minutes after the peak value of the particular thermal stimulus or the stimulus thresholds R_{G1} . . . R_{G5}. In the case of the salivary amylase value S₅₂, an increased value is measured, it being possible to infer therefrom that the patient senses a certain level of pain. Since the course of the thermal stimulus cycle is always the same, it can be inferred. therefrom that the patient, has become more pain-sensitive, or that the effect of the analgesic has subsided. The inventive device 1 comprises an output device, for example a screen 5b on which the salivary amylase value S(t) or a value dependent on the salivary amylase value is displayed. A value dependent on the salivary amylase value S(t) is to be understood to mean any variable giving the physician feedback about the state of the patient, said value always being based on the measured salivary amylase value S(t). Such a value dependent on the salivary amylase value S(t) could, for example, also be an alarm signal such as a red flashing which is triggered when the salivary amylase value S(t) exceeds a predeterminable reference value S_{R}, it being possible for the alarm signal A1 to be displayed, for example, on a display 5d for alarm values. Upon occurrence of an increased salivary amylase value S(t) or of an alarm value A1, a physic tan can take the necessary measures on the basis of his expert knowledge and, for example, administer an analgesic M₂ or, for example, abandon the operation. Toward the end of the operation, a physician can also deliberately allow the rise in the salivary amylase value S(t) in order to initiate the wake-up phase as early as during the operation and in order, as a result, to shorten the duration of the subsequent wake-up phase 21d. In the example depicted in FIG. 8, an analgesic M₂ is administered, the result of this being that the subsequent salivary amylase values S₅₃ to S₆₀ indicate that the patient does not sense any pain.

FIG. 9 shows the thermal stimulus depicted in FIG. 8 or thermal stimulus cycle R₃ with stimulus threshold. R_{G3} in detail. In FIG. 8, the salivary amylase value S(t) was measured at discrete times every six minutes in order to determine the salivary amylase values S₅₀ . . . S₆₀. FIG. 9 shows an example in which the salivary amylase value is measured at short intervals, for example every 10 seconds, and so what is displayed is not a single salivary amylase value S₅₂ but, in detail, the course of the salivary amylase value S₅₂ as a function of time. The time delay Δts between the exert on of the stimulus threshold R_{G3} and the occurrence of the maximum salivary amylase value S_{Max} is preferably known from the measurement of sensitivity to pain and is, for example, 1 to 3 minutes, it being possible for the maximum salivary amylase value S_{Max} to occur within a time frame of Δtz of, for example, ±2 minutes after the time delay Δts. The maximum salivary amylase value S_{Max} which occurred in FIG. 9 is within said time frame, and so it can be inferred therefrom that the maximum salivary amylase value S_{Max} of the salivary amylase value course S₅₂ was caused by the thermal stimulus or thermal stimulus cycle R₃ or by the stimulus threshold R_{G3}. The salivary amylase value described by S₅₂ in FIG. 8 corresponds, in FIG. 9, to the maximum salivary amylase value S_{Max}.

FIG. 10 shows the thermal stimulus R₃ with stimulus threshold R_{G3}, as depicted in FIG. 8, and also two exemplary courses of salivary amylase values Sₓ, S_{y} in detail, the salivary amylase values being measured at short intervals, for example every 10 seconds, in this example. The course of the salivary amylase value Sₓ already has a slope before the thermal stimulus R₃, and has the maximum value outside the time frame of Δtz, it being possible to infer therefrom that said salivary amylase value course Sₓ was not caused by the thermal stimulus R₃. The course of the salivary amylase value S_{y} has a slope only after a relatively lone T period after the thermal stimulus R₃, and has the maximum value outside the time frame of Δtz, it again being possible to infer therefrom that said salivary amylase value course S_{y} was not caused by the thermal stimulus R₃. Such a situation arises during an operation by, for example, a severe level of pain being inflicted on the patient as a result of the surgical action. Such a situation can occur in particular during analgosedation. If a high salivary amylase value is established outside the time frame Δtz, said value is also displayed via the screen 5b or the display 5d for alarm values in order to signal this emergency state to the physician. As depicted in FIG. 10, it may prove to be advantageous to also monitor the slope α with respect to the salivary amylase value S(t), especially in the case of strongly increasing salivary amylase values S(t). For example, a maximum slope value α_{Max} could be predetermined as a reference value, with a signal such as, for example, an alarm value being triggered when said maximum slope value α_{Max} is exceeded. This makes it possible to capture at an early stage a strong rise in the salivary amylase value S(t) before said value has reached the maximum value and so a physician is informed very quickly about the severe pain that has acted on the patient. Advantageously, a physician can respond very quickly in such a situation by, for example, briefly interrupting the pain-causing action during the operation, proceeding more gently, or administering an additional analgesic.

FIGS. 11 and 12 show thermal stimuli R₁ . . . R₃ with stimulus threshold R_{G1} . . . R_{G3} and the corresponding salivary amylase values S(t) S₆₁, S₆₂, S₆₃, or S₆₄, S₆₅, S₆₆, as a function of time. Said figures show, by way of example, monitoring of a patient during analgosedation. One analgesic is dosed such that the patient still lightly responds to the thermal stimuli R₁ . . . R₃, i.e., feels a low level of pain which is, however, very easily tolerable. Compared to FIG. 11, the same thermal stimuli R₁ . . . R₃ are also exerted in. FIG. 12, the salivary amylase values S(t) S₆₄, S₆₅, S₆₆ having a higher maximum value S_{Max}. Therefore, the thermal stimuli R₁ . . . R₃ is causing more severe pain, or the patient'sensation of pain is less attenuated, more particularly the sensation of pain is not completely suppressed. On the basis of the thermal stimuli R₁ . . . R₃ depicted in FIGS. 11 and 12 and the measured maximum values S_{Max} of the salivary amylase values S(t), it is possible for a physician to monitor, assess and also control the depth of analgosedation during the operation.

FIG. 13 shows a salivary amylase value S(t) as a function of time within the period T₃ to T₃+60 minutes, the analgosedation being managed in an intermittent manner such that certain levels of pain acting on the patient are allowed. At measurement point S₇₂, a relatively large rise in the salivary amylase value S(t) is established, whereupon an analgesic N1 is administered. Consequently, measurement points S₇₃ and S₇₄ stabilized, although a further increase is established at measurement point S₇₅, and so an analgesic N₂ is administered. After a further rise takes place in measurement point. S₇₆ and a very strong rise takes place in measurement point S₇₇, another, stronger-acting analgesic M₃ administered, whereupon measurement points S₇₈, S₇₉, S₈₀ have very low values. FIG. 13 therefore shows an example of how sedation and also anesthesia can be managed during the operation using the device according to the invention or the method according to the invention. In particular, it is also possible to identify anesthesia awareness of a patient with sensation of pain and to respond appropriately thereto.

FIG. 14 shows a further exemplary embodiment of a device 1 for noninvasively monitoring a sedated or anesthetized patient, the device 1 additionally further comprising a dispensing device 8 for dispensing a medicament, in contrast to the embodiment depicted in FIG. 1. This enables, for example, a physician to select a medicament and to set a dispensing range such that the device 1 automatically dispenses the selected medicament according to the salivary amylase value S(t) or a value derived therefrom and according to the boundary conditions predetermined by the physician.

FIG. 15 shows the example of a measurement of the individual sensitivity to pain of one person, as already depicted and described in FIG. 4. The thermal stimulator 7 is applied to the skin of the patient, for example on the lower arm, and the temperature is, starting at 25° C., increased by 1° C. every 2 seconds until the person finds the pain intolerable, and so the temperature increase is stopped, at 48° C. in the depicted example in contrast to the approach depicted in FIG. 4, the person additionally subjectively rates the sensed pain stimulus, for example on a scale from 0 to 10. To this end, the person, using a free hand, can move, for example, a linearly slidable input means 9, and thus adjust the subjectively sensed pain intensity, preferably continuously, according to the rising temperature. FIG. 15 shows, by means of the points P₁ . . . P₈, the pain intensity on a scale from 0 to 10 that was subjectively sensed by a person in the course of the temperature increase, the person aborting the measurement by choice at the value R_{Max} of 48° C. The rise in the salivary amylase values takes place, as already described in FIG. 4, with a time delay Δts. From the two curve profiles of the point P(i) or P₁ . . . P₈ and of the salivary amylase values S₂₀ . . . S₂₈, it is now possible to determine a relationship between a measured salivary amylase value S(t) and the subjectively sensed. pain intensity P(i). If this relationship between salivary amylase values S(t) and the subjectively sensed pain intensity P(i) is measured and stored before an operation, it is possible during the operation not only to depict the current salivary amylase value S(t), but also to additionally calculate, output and depict the subjective pain intensity P(i). The device according to the invention or the method therefore makes it possible to calculate and output the subjective pain intensity P(i) of a sedated or anesthetized patient. As a result, a physician during a surgical procedure on the human body, for example an operation, is provided with information about the patient's acute, individual sensation of pain. This allows, for example, an individual pain management or anesthesia management, a personalized administration of medicaments, and a personalized wake-up phase after the operation.

FIG. 16 shows the example of a measurement of the individual sensitivity to pain of one person, as already depicted and described in FIG. 4. In. FIG. 16, the curve profile of this first person is referred to as O₁, the maximum temperature stimulus as R_{1Max}, and the maximum salivary amylase value as S_{1Max}. In FIG. 16, the individual sensitivity to pain of a second person is now additionally depicted. In FIG. 16, the curve profile of said second person is referred to as O₂, the maximum temperature stimulus as R_{2Max}, and the maximum salivary amylase value as S_{2Max}. Compared to the first person O₁, the second person O₂ has a higher sensitivity to pain, and this can be seen from the fact that the second person O₂ has already interrupted the measurement at a lower maximum temperature stimulus and that, as is evident from the curve profile O₂, the second person has a higher salivary amylase value at said temperature stimulus R_{2Max} than the first person, as is evident from the curve profile O₁. The method. according to the invention or the device according to the invention makes it possible to capture these individual differences and also to take them into account during the monitoring of the sedated or anesthetized patient. Preferably, the stimulus threshold R_{G} of the pain stimulus R during the operation 21c and preferably also before it and after it is selected such that the stimulus threshold R_{G} corresponds to the maximum temperature stimulus R_{Max} of the corresponding patient. In the case of the two aforementioned patients, the stimulus threshold R_{G} would therefore be R_{1Max} or R_{2Max}. This makes it possible to easily determine the depth of sedation or general anesthesia, as will be explained below. From the measurement of sensitivity to pain, the pair of values S_{1Max} and R_{1Max} is known for the first patient. Assuming the stimulus threshold R_{G1}, R_{G2}, R_{G3} in FIGS. 11 and 12 has the value R_{1Max}, it is possible by measuring the maximum value S_{Max} of each salivary amylase course S₆₁, S₆₂, S₆₃, S₆₄, S₆₅, S₆₆ to immediately infer the depth of sedation or general anesthesia. In the absence of sedation or general anesthesia, the measured salivary amylase value would have to have the value S_{1Max}. Owing to the sedation or general anesthesia, the measured salivary amylase value has a lower value. Therefore, it is easily possible to determine the effect of the administered medicaments or the current depth of sedation or general anesthesia, personalized to the particular patient. The stimulus threshold R_{G} can naturally also be selected lower than the maximum temperature stimulus R_{Max}, for example as a percentage of the maximum temperature stimulus R_{Max}, and so the stimulus threshold R_{G} is, for example, 50%, or 80% or 90%, of the maximum temperature stimulus R_{Max}.

In the preceding examples, the controllable pain stimulus was always described as thermal stimuli. In the preceding examples, the thermal stimuli or the thermal stimulus cycles R₁ . . . R₃ were always generated at regular intervals, i.e., periodically. It is also possible to administer the thermal stimuli or the thermal stimulus cycles only during a temporal subsection of the operation 21c. It is also possible to allow the thermal stimuli or the thermal stimulus cycles to act on the patient only during the sedation or general anesthetization phase 21b in order to monitor the depth or the progression of sedation or of general anesthesia in said phase. It is also possible to vary the repetition rate of the thermal stimuli or of the thermal stimulus cycles in order, for example, to capture, during a critical phase of an operation 21c, the sensation of pain or the depth of sedation or of anesthesia at shorter intervals. It is also possible for the thermal stimulus or the thermal stimulus cycle to be triggered only sporadically, for example manually by the physician, in order to check in certain phases of an operation 21c the depth of sedation or of anesthesia.

However, the device according to the invention or the method according to the invention is also suitable for monitoring local anesthesia or minimal sedation. For instance, this could, for example in the case of a dentist for local anesthesia in the mouth, or for example in the case of local anesthesia of a limb, e.g., a leg, be effected such that the thermal stimulator is applied to the skin of the body part to be anesthetized, thermal stimuli are delivered, and salivary amylase values S(t) are measured. The salivary amylase values S(t) and/or the subjective pain intensity P(t) derived therefrom can be outputted and. displayed, and so a physician can identify from which point in time the local anesthesia is having an effect, and/or can identify whether the local anesthesia is sufficiently deep, and/or can identify whether the local anesthesia is subsiding. An advantage of such a procedure can be seen in that the local anesthesia can be individually managed and monitored, that the local anesthesia is carried out only to the depth that is required, that unnecessary pain can be avoided, and that the local anesthesia lasts only as long as is required for the surgical procedure.

The device according to the invention or the method according to the invention is in particular also suitable for treating emergency patients. In the case of emergency operations, it is usually necessary to dispense with a general-anesthesia preparation, so that the surgical procedure can start as quickly as possible. Such emergency patients are usually also not on an empty stomach. In such cases, the device according to the invention or the method according to the invention makes it possible, for example, to carry out analgosedation in which it is ensured that the patient does not sense any pain. In such cases, it is frequently not possible to carry out before the operation an individual clarification of sensitivity to pain, as depicted in FIGS. 4 and 15. In such a case, a data set characteristic of the patient is advantageously selected from an existing data collection, for example on the basis of selection criteria such as gender, weight, age, body type, etc., in order to carry out the monitoring of the sedated or anesthetized patient on the basis of said characteristic data set.

In FIGS. 6 and 8 to 12, the thermal stimulus cycles R, R₂, R₃ are, by way of example, depicted as proceeding symmetrically as a function of time, having identical and constant rates of rise and drop. However, the thermal stimulus cycles R₁, R₂, R₃ can also have other curve profiles as a function of time for obtaining a defined, predetermined stimulus threshold R_{G1}, R_{G2}, R_{G3} . . . The rate of drop of the thermal stimulus cycles R₁, R₂, R₃ can, after attainment of the stimulus thresholds R_{G1}, R_{G2}, R_{G3}, also proceed much more flatly and, in particular, also nonlinearly, especially when the pain-stimulus generation device 7 does not comprise a Peltier element. Moreover, it is also possible to control the pain-stimulus generation device 7 such that at least some of the stimulus thresholds R_{G1}, R_{G2}, R_{G3} have different temperature values, for example when a lower temperature value is sufficient for generating and measuring a salivary amylase value S₅₀, S₅₁, S₅₂ . . . The invention uses a rise in the salivary amylase value S(t), as measured as a response to a thermal pain stimulus R, or the maximum amylase value S_{Max} of the salivary amylase value S(t), as measured after the thermal pain stimulus R, of a sedated or anesthetized person 10 in order to determine the depth of sedation or the depth of general anesthesia. In this connection, the thermal pain stimulus R rises up to a stimulus threshold Rg and then drops. The pain stimulus R is preferably a succession of stimuli which rise up to a stimulus threshold Rg and then drop. Advantageously, a rise in the salivary amylase value S(t) is registered. within a time frame from 10 seconds to 300 seconds after the stimulus threshold Rg.

1-25. (canceled)

26. A device (1) for noninvasively monitoring a sedated or anesthetized person (10), comprising a pain-stimulus generation device (7), wherein the pain-stimulus generation device (7) generates a thermal stimulus, and also comprising a control device (4), wherein the control device (4) is designed such that it controls the pain-stimulus generation device (7) in order to generate a pain stimulus (R), characterized by
a measurement device (2) for capturing a salivary amylase value (S(t)) of the person (10), and also an output device (5), wherein the control device (4) is designed such that it, after effected generation of the pain stimulus (R), captures the salivary amylase value (S(t)) and outputs an output value (A) via the output device (5), wherein the output value (A) is the salivary amylase value (S(t)) or a value dependent on the salivary amylase value (S(t)).

27. The device as claimed in claim 26, characterized in that the control device (4) is designed such that it increases the pain stimulus (R) generatable by the pain-stimulus generation device (7) at intervals over time up to a stimulus threshold (R_{G}) and then reduces it.

28. The device as claimed in claim 26, characterized in that the pain-stimulus generation device (7) comprises a heatable device (7b) which can be contacted with the skin.

29. The device as claimed in claim 26, characterized in that the pain-stimulus generation device (7) comprises an electrically heatable device (7b) and also a cooling device, so that the thermal stimulus delivered by the pain-stimulus generation device (7) to the person has a temperature course as a function of time that is predetermined by the control device (4).

30. The device as claimed in claim 26, characterized in that the thermal stimulus generated by the pain-stimulus generation device (7) has a stimulus threshold (Rg), that the control device (4) has a time frame within which the salivary amylase value (S(t)) is captured, wherein the time frame opens at an opening time (tₘᵢₙ), and wherein the opening time (tₘᵢₙ) is within a range from 10 seconds to 60 seconds after the occurrence of the stimulus threshold (Rg).

31. The device as claimed in claim 26, characterized in that the control device (4) controls the pain-stimulus generation device (7) such that the thermal stimulus rises at a rate within the range between 0.5° C./sec and 2° C./sec, and preferably at 1° C./sec.

32. The device as claimed in claim 6, characterized in that the control device (4) controls the pain-stimulus generation device (7) such that the thermal stimulus, after attainment of the stimulus threshold (R_{G}), lowers at a rate within the range between 0.5° C./sec and 2° C./sec, and preferably at 1° C./sec.

33. The device as claimed in claim 26, comprising a storage device (3) for storing at least one reference value (S_{R}), wherein the control device (4) is designed such that it outputs an alarm value (A1) as an output value (A) if the salivary amylase value (S(t)) exceeds the reference value (S_{R}).

34. The device as claimed in claim 26, characterized in that it comprises a dispensing device (8) for the dosed dispensing of a sedating, analgesic and/or general-anesthetizing active ingredient (N,M), wherein the control device (4) controls the dispensing device (8) such that the dosage is effected according to the salivary amylase value (S(t)).

35. A method for noninvasively monitoring a sedated or anesthetized person (10), by delivering a pain stimulus (R) to the person (10), by measuring a salivary amylase value (S(t)) of the person (10) after effected delivery of the pain stimulus (R), and by outputting the measured salivary amylase value (S(t)) or a value dependent on the salivary amylase value (S(t)), wherein the pain stimulus (R) generated by the pain-stimulus generation device (7) is increased up to a stimulus threshold (R_{G}) in a repeated manner and is then reduced, wherein a temperature stimulus is used as pain stimulus (R).

36. The method as claimed in claim 35, characterized in that the temperature of the pain stimulus (R) is increased and reduced at regular intervals, having a cycle duration within the range between 1 to 15 minutes.

37. The method as claimed in claim 35, characterized in that a reference value (S_{R}) is predetermined for the salivary amylase value (S(t)), and that an output value (A) is generated if the salivary amylase value (S(t)) exceeds the reference value (S_{R}).

38. The method as claimed in claim 35, characterized in that the control device (4) has a time frame within which the salivary amylase value (S(t)) is captured, and that the opening time (t,) of the time frame, from which the salivary amylase value (S(t)) is captured, is within a range from 10 seconds to 60 seconds after the occurrence of the stimulus threshold (Rg).

39. The method as claimed in claim 35, characterized in that the pain stimulus (R) rises at a rate within the range between 0.5° C./sec and 2° C./sec, and preferably at 1° C./sec.

40. The method as claimed in claim 35, characterized in that the pain stimulus (R) lowers at a rate within the range between 0.5° C./sec and 2° C./sec, and preferably at 1° C./sec.

41. The method as claimed in claim 35, that an individual sensitivity to pain is captured prior to the monitoring, by exposing the person to at least one maximum pain stimulus value (R_{Max}) and preferably to an increasing pain stimulus (R), and wherein the salivary amylase value (S(t)) occurring in connection with the pain stimulus (R) is measured, and wherein the particular value of the pain stimulus (R) and the corresponding salivary amylase value (S(t)) is stored.

42. A method for operating a device as claimed in claim 26, characterized in that a dispensing device (8) is controlled via the output value (A) such that a sedating, analgesic or general-anesthetizing active ingredient is dispensed according to the output value (A) and/or the dosage of the sedating, analgesic or general-anesthetizing active ingredient is increased or reduced.
